(19) United States
(12) Patent Application Publication
(10) Pub. No.: US 2026/0033766 A1
(43) Pub. Date: Feb. 5, 2026

(54) NEURAL PROBE, NEURAL PROBE INTERFACE DEVICE CONNECTED TO THE NEURAL PROBE, AND BIOLOGICAL EXPERIMENT SYSTEM INCLUDING THE NEURAL PROBE AND THE NEURAL PROBE INTERFACE DEVICE

(71) Applicant: ELECTRONICS AND TELECOMMUNICATIONS RESEARCH INSTITUTE, Daejeon (KR)

(72) Inventors: Myungjoon Kwack, Daejeon (KR); Jaegyu Park, Daejeon (KR)

(21) Appl. No.: 19/287,587

(22) Filed: Jul. 31, 2025

(30) Foreign Application Priority Data
Aug. 1, 2024 (KR) 10-2024-0102792
Jul. 30, 2025 (KR) 10-2025-0103789

(51) Int. Cl. A61B 5/293; A61B 5/263; A61B 5/377

(52) U.S. Cl. CPC A61B 5/293; A61B 5/263; A61B 5/377; A61B 2503/42

(57) ABSTRACT

Disclosed are a neural probe, a neural probe interface device connected to the neural probe, and a biological experiment system including the neural probe and the neural probe interface device. The neural probe collects a neural signal generated from neurons based on an optogenetics technique. The neural probe includes a neural probe substrate, an optical input port disposed in the neural probe substrate and configured to receive an optical signal from the outside, an optical output port disposed in the neural probe substrate and configured to transmit the optical signal to neurons, a first electrode configured to receive the neural signal, and a second electrode configured to receive the neural signal from the first electrode through an electric wire and to transmit the neural signal to the outside by using any one of a wired communication method and a wireless communication method.

### CROSS-REFERENCE TO RELATED APPLICATIONS

This application claims priority under 35 U.S.C. § 119 to Korean Patent Application Nos. 10-2024-0102792, filed on Aug. 1, 2024, and 10-2025-0103789, filed on Jul. 30, 2025, the disclosure of which is incorporated herein by reference in its entirety.

### BACKGROUND

### 1. Technical Field

The present disclosure relates to a neural probe that stimulates or suppresses neurons and extracts a neural signal from neurons by using an optogenetics technique, an interface device for applying the neural probe to biological experiments, and a biological experiment system including the neural probe and the interface device.

### 2. Related Art

In general, the transfer of the signal of a neuron is performed by an electrochemical action. Conventionally, in order to artificially activate a nerve, a method of making an electrode to be adjacent to neurons and applying the right amount of a current or a voltage to the nerve is used. Furthermore, when protein that has a light-reactive ion channel action like channel rhodopsin-2 (ChR2) is transplanted on neurons and light having a specific wavelength is radiated to the protein, a nerve may be activated even by photic simulation not by an electric stimulus. An optogenetics technique in which only a specific type of nerve can be stimulated or suppressed by using light as described above was published in 2005. In the existing electric stimulus method, it was not possible to identify the specific role of each nerve within a living organism. However, with the advent of optogenetic technology, it has become possible to investigate the functions of nerves at the cellular level using optogenetics, thereby opening a new horizon in neuroscience research.

In general, for optogenetics experiments, a method of connecting an external light source to an optical fiber, making the end of the optical fiber adjacent to neurons by using the optical fiber as a medium, and radiating light to a nerve is adopted. However, due to the manufacturing limitations of optical fibers, it is difficult to radiate light to regions other than the end. This poses significant limitations in the study of neural tissues, which have a three-dimensional structure composed of densely bundled cells.

As part of efforts to overcome such a problem, photonics neural probe technique research in which an optical output port capable of outputting an optical signal is disposed in multiple optical waveguides and ends thereof so that light is radiated to multiple cell tissues to be radiated by using an optical semiconductor-based photonics technique is actively performed. In addition, a technique for recording a neural signal is required in addition to a technique for stimulating or suppressing a nerve by using light. A signal that is generated when a brain nerve is activated is an electrophysiologic signal. In order to measure the electrophysiologic signal, it is necessary to make an electrode to be directly adjacent to the brain nerve. Accordingly, in order to simultaneously realize the stimulation of the nerve and the recording of the neural signal, the existing electrode-based neural probe technique is grafted onto a photonics neural probe.

In order to use an optogenetics neural probe including an electrode capable of both the stimulation or suppress of a nerve and the recording of an electrical neural signal using light as described above, an electrical connection between a signal analyzer and the electrode of a neural probe is required along with the optical interconnection of an external light source and the neural probe. Accordingly, there is a technical problem in terms of a module and a system for implementing the requirement.

The background technique relates to contents that are owned or obtained by an inventor in a process of deriving the present disclosure, and it cannot be essentially sure that the background technique is a known technique disclosed in the general public prior to the application of this applicant.

### PRIOR ART DOCUMENT

### Patent Document

(Patent Document 1) KR 10-1828149 B1 (issued on Feb. 5, 2018)
(Patent Document 2) U.S. Pat. No. 10,471,273 B2 (issued on Nov. 12, 2019)

### SUMMARY

Various embodiments are directed to providing a neural probe including a plurality of optical input and output ports and a plurality of electrodes, and an interface device for applying the neural probe to biological experiments and simultaneously implementing the optogenetics-based neural stimulation and inhibition, and the electrode-based neural signal recording.

Objects of the present disclosure are not limited to the aforementioned object, and other objects not described above may be evidently understood by those skilled in the art from the following description.

A neural probe according to an embodiment of the present disclosure collects a neural signal generated from neurons based on an optogenetics technique. The neural probe includes a neural probe substrate, one or more optical input ports disposed in the neural probe substrate and configured to receive an optical signal from the outside, one or more optical output ports disposed in the neural probe substrate and configured to transmit the optical signal to neurons, one or more first electrodes configured to receive the neural signal, and one or more second electrodes configured to receive the neural signal from the first electrode through an electric wire and to transmit the neural signal to the outside by using any one of a wired communication method and a wireless communication method.

In an embodiment of the present disclosure, the neural probe substrate includes a base section and a protrusion part. Furthermore, the thickness of the protrusion part is the thickness or less of the base section.

In an embodiment of the present disclosure, the optical input port is mounted on the base section.

In an embodiment of the present disclosure, the optical output port is mounted on the protrusion part.

In an embodiment of the present disclosure, the first electrode is mounted on the protrusion part.

In an embodiment of the present disclosure, the second electrode is mounted on the base section.

In an embodiment of the present disclosure, the neural probe further includes an optical waveguide disposed in the neural probe substrate and configured to transmit the optical signal to the optical output port.

In an embodiment of the present disclosure, the optical waveguide includes any one material, among SiN, SiO₂, SiON, polymer, SU-8, and parylene.

In an embodiment of the present disclosure, the optical waveguide and the electric wire are disposed at different heights in relation to the neural probe substrate.

In an embodiment of the present disclosure, the neural probe further includes an optical switch circuit disposed in the neural probe substrate and configured to transmit the optical signal to the optical output port. The optical switch circuit dynamically changes an optical interconnection between the optical input port and the optical output port in response to a control signal.

A neural probe interface device according to an embodiment of the present disclosure receives a neural probe for collecting a neural signal generated from neurons based on an optogenetics technique and that is connected to the neural probe.

The neural probe includes a neural probe substrate, one or more optical input ports disposed in the neural probe substrate and configured to receive an optical signal from the outside, one or more optical output ports disposed in the neural probe substrate and configured to transmit the optical signal to neurons, one or more first electrodes configured to receive the neural signal, and one or more second electrodes configured to receive the neural signal from the first electrode through an electric wire and to transmit the neural signal to the outside by using any one of a wired communication method and a wireless communication method.

The neural probe interface device includes a neural probe holder configured to receive and fix the neural probe, an electrode connection part connected to the second electrode and configured to receive the neural signal from the second electrode, and an external electrode interface configured to transmit the neural signal to an external signal analyzer or a computer.

In an embodiment of the present disclosure, the neural probe interface device further includes an optical connector configured to receive an optical signal for neural stimulation or suppression from an external light source and to make the optical signal incident on the optical input port of the neural probe through an optical fiber.

In an embodiment of the present disclosure, the neural probe interface device further includes an optical element array comprising optical elements aligned at predetermined intervals. An optical signal emitted from the optical element is incident on the optical input port of the neural probe.

A biological experiment system according to an embodiment of the present disclosure is a system that performs biological experiments by using a neural probe that collects a neural signal generated from neurons based on an optogenetics technique. The biological experiment system includes a light source configured to output an optical signal, a signal generator configured to output the optical signal in a form of a pulse at predetermined interval, an optical switch configured to transmit the optical signal by selecting one or more of optical input ports of the neural probe by setting, a neural probe configured to receive the optical signal, transmit the optical signal to neurons of a biological experiment target, and receive the neural signal from the neurons, a data acquisition system configured to collect the neural signal, and a computer configured to generate results of analysis of an active pattern by analyzing the neural signal collected by the data acquisition system.

In an embodiment of the present disclosure, the biological experiment device further includes an attenuator configured to adjust an intensity of the optical signal output from the light source.

In an embodiment of the present disclosure, the biological experiment device further includes a polarization controller configured to control polarization of the optical signal output from the light source.

In an embodiment of the present disclosure, the biological experiment device further includes an optical fiber array connector configured to receive the optical signal from the optical switch through a selected optical fiber and a neural probe interface device connected to the optical fiber array connector and configured to transmit the optical signal received from the optical fiber array connector from the optical switch to an optical input port of the selected neural probe.

In an embodiment of the present disclosure, the neural probe includes a neural probe substrate, one or more optical input ports disposed in the neural probe substrate and configured to receive the optical signal, one or more optical output ports disposed in the neural probe substrate and configured to transmit the optical signal to neurons, one or more first electrodes configured to receive the neural signal, and one or more second electrodes configured to receive the neural signal from the first electrode through an electric wire and to transmit the neural signal to the outside by using any one of a wired communication method and a wireless communication method.

In an embodiment of the present disclosure, the neural probe interface device included in the biological experiment system includes a neural probe holder configured to receive and fix the neural probe, an electrode connection part configured to receive a neural signal collected from the biological experiment target by the neural probe from a second electrode of the neural probe, and an external electrode interface configured to transmit the neural signal to an external signal analyzer or a computer.

A neural probe device according to an embodiment of the present disclosure includes a neural probe and an interposer physically combined with the neural probe and optically and electrically connected to the neural probe.

The neural probe includes a neural probe substrate, one or more optical input ports disposed in the neural probe substrate and configured to receive an optical signal for neural stimulation or suppression, one or more optical output ports disposed in the neural probe substrate and configured to transmit the optical signal to neurons, one or more first electrodes configured to receive a neural signal from the neurons, and one or more second electrodes configured to receive the neural signal from the first electrode through an electric wire and to transmit the neural signal to the outside by using any one of a wired communication method and a wireless communication method.

The interposer is constructed by mounting an optical connector, an optical fiber, a fourth electrode, and an electrode connector on any one of a printed circuit board (PCB) neural probe substrate and a glass-based neural probe substrate.

The optical connector receives the optical signal from an external light source and transmits the optical signal to the optical fiber.

The optical fiber makes the optical signal incident on the optical input port through an end of the optical fiber.

The fourth electrode is electrically connected to the second electrode and transmits the neural signal to the electrode connector.

The electrode connector transmits the neural signal to any one of an external terminal and an external analysis system.

According to an embodiment of the present disclosure, there is provided the interface device, which is necessary for the neural probe including the plurality of optical input and output ports and a plurality of electrodes and can implement both optical interconnection and an electrical connection efficiently.

Furthermore, according to an embodiment of the present disclosure, it is possible to replace only a neural probe that is difficult to be permanently used due to a damage possibility in a process of being inserted into a living body with a new probe and to reduce a cost for experiments because the interface device and a system that operates in conjunction with the interface device can be consistently used.

Effects of the present disclosure which may be obtained in the present disclosure are not limited to the aforementioned effects, and other effects not described above may be evidently understood by a person having ordinary knowledge in the art to which the present disclosure pertains from the following description.

### BRIEF DESCRIPTION OF THE DRAWINGS

FIGS. 1A and 1B are front views of a neural probe including a plurality of optical output ports and a plurality of electrodes.

FIG. 2 is a diagram illustrating an embodiment of a neural probe in which an optical switch circuit is integrated.

FIG. 3 is a schematic diagram of a cross section of a neural probe.

FIGS. 4A to 4C are diagrams illustrating a construction of an optical fiber-based neural probe interface device.

FIGS. 5A and 5B are diagrams illustrating a construction of an optical element-based neural probe interface device.

FIGS. 6A and 6B are diagrams illustrating the coupling of a neural probe and a neural probe interface device.

FIG. 7 is an exemplary diagram of a biological experiment system using the neural probe.

FIGS. 8A and 8B are diagrams illustrating examples of a neural probe device having a form in which the neural probe and an interposer are combined.

### DETAILED DESCRIPTION

Advantages and characteristics of the present disclosure and a method for achieving the advantages and characteristics will become apparent from embodiments described in detail later in conjunction with the accompanying drawings. However, the present disclosure is not limited to the disclosed embodiments, but may be implemented in various different forms. The embodiments are merely provided to complete the present disclosure and to fully notify a person having ordinary knowledge in the art to which the present disclosure pertains to the category of the present disclosure. The present disclosure is merely defined by the category of the claims. Terms used in this specification are used to describe embodiments and are not intended to limit the present disclosure. In this specification, an expression of the singular number includes an expression of the plural number unless clearly defined otherwise in the context. The term “comprises” and/or “comprising” used in this specification does not exclude the presence or addition of one or more other components, steps, operations and/or components in addition to mentioned components, steps, operations and/or components.

Terms, such as a first and a second, may be used to describe various components, but the components should not be restricted by the terms. The terms may be used to only distinguish one component from the other components. Accordingly, a first component may be named a second component without departing from the scope of a right of the present disclosure. Likewise, a second component may also be named a first component.

When it is described that one component is “connected” or “coupled” to the other component, it should be understood that one component may be directly connected or coupled to the other component, but a third component may exist between the two components. In contrast, when it is described that one component is “directly connected to” or “directly coupled to” the other component, it should be understood that a third component does not exist between the two components. Other expressions for describing relations between components, that is, “between ˜”, “just between ˜”, “adjacent to ˜”, and “neighboring ˜”, should be likewise construed.

In describing the present disclosure, a detailed description of a related known technology will be omitted if it is deemed to make the subject matter of the present disclosure unnecessarily vague.

Hereinafter, embodiments of the present disclosure are described in detail with reference to the accompanying drawings. In describing the present disclosure, in order to facilitate general understanding of the present disclosure, the same reference numeral is used for the same mean regardless of the reference numeral.

An optogenetics neural probe (hereinafter referred to as a “neural probe”) according to an embodiment of the present disclosure transmits an optical signal to neurons or receives an electrical signal from neurons.

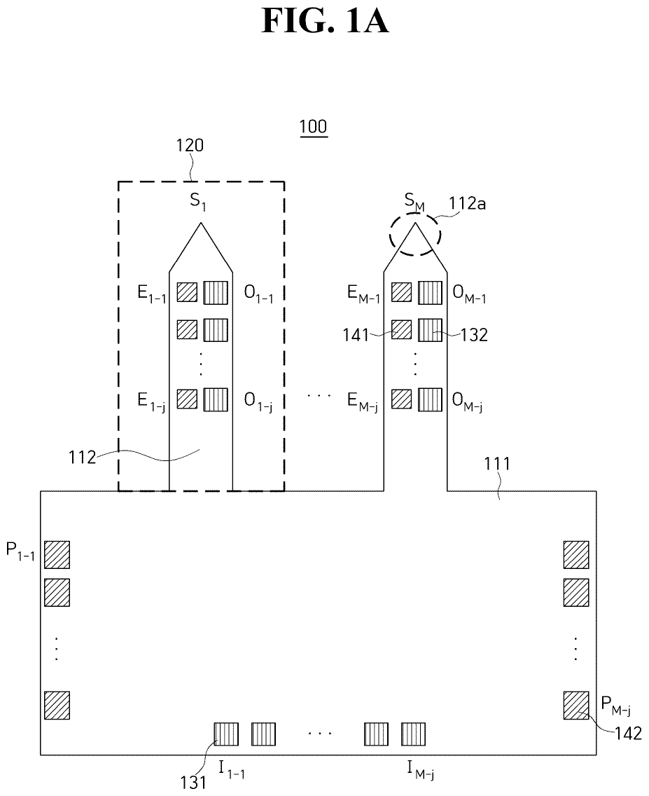

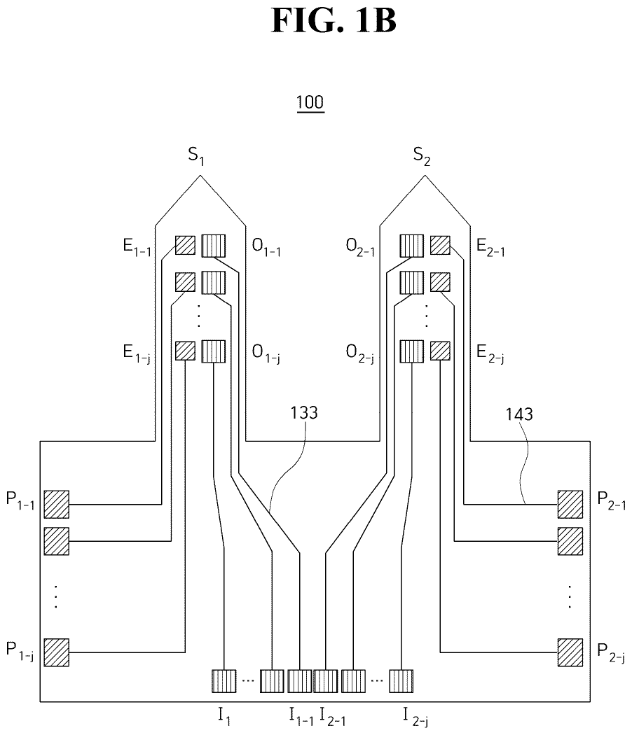

FIGS. 1A and 1B are front views of a neural probe including a plurality of optical output ports and a plurality of electrodes. FIG. 1A is a diagram illustrating an example in which the number of probe parts is M in the neural probe. FIG. 1B is a diagram illustrating an example in which the number of probe parts is two in the neural probe.

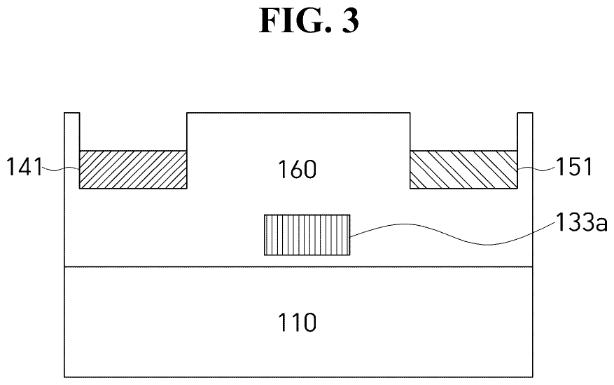

A neural probe 100 according to an embodiment of the present disclosure may be implemented in a form in which one or more optical signal ports 130 (not illustrated) and one or more electrodes have been mounted on a neural probe substrate 110 (refer to FIG. 3). The neural probe substrate 110 includes a base section 111 and a protrusion part 112. The number of protrusion parts 112 included in one neural probe 100 may be plural. In the embodiment of FIG. 1A, the number of protrusion parts 112 is M. In order to distinguish between the protrusion parts 112, symbols S₁ to S_{M} are assigned to the protrusion parts 112, respectively. Furthermore, in the embodiment of FIG. 1B, the number of protrusion parts 112 is two. Symbols S₁ and S₂ are assigned to the protrusion parts 112, respectively.

The protrusion part 112 refers to an area that is inserted into neurons. In this specification, a sharp probe structure of the protrusion part 112, which is inserted into neurons, is denoted as a probe peak part 112a.

The base section 111 is an area except the protrusion parts 112 in the entire area of the neural probe substrate 110. A part of or the entire base section 111 may be received in a neural probe interface device 200, 300, 470 to be described later.

The optical signal port 130 is divided into an optical input port 131 and an optical output port 132. In the neural probe 100 according to the embodiment of FIGS. 1A and 1B, a plurality of optical input ports 131 is mounted on the base section 111, and a plurality of optical output ports 132 is mounted on the protrusion part 112. Furthermore, in the neural probe 100, a first electrode 141, that is, an electrode for extracting a neural signal, is mounted on the protrusion part 112. A second electrode (hereinafter also denoted as an “electrode pad”) 142, that is, an electrode that transmits a neural signal extracted by the first electrode 141 to the outside, is mounted on the base section 111. The neural probe 100 according to the embodiment of FIG. 1B includes two protrusion parts 112 (M=2). In the embodiment of FIG. 1B, a plurality of optical input ports 131 (I_{M-j}) and a plurality of optical output ports 132 (O_{M-j}) are connected by an optical waveguide 133. The optical input port 131 transmits an optical signal that is mounted on the neural probe 100 or that is emitted from an external optical element to the optical waveguide 133. The optical signal is transmitted to neurons through the optical waveguide 133 and the optical output port 132. The first electrode 141 receives an electrophysiological signal (hereafter referred to as a “neural signal”) generated from neurons, and transmits the neural signal to the second electrode 142 through an electric wire 143. The neural signal may be a signal that is generated by an optical signal, but may be a signal that is autonomously generated from neurons without the transmission of an optical signal.

Furthermore, a plurality of first electrodes 141 (E_{M-j}) and a plurality of second electrodes 142 (P_{M-j}) may be electrically connected through the electric wires 143.

In this specification, a connection through an optical waveguide (e.g., 133 in FIG. 1B) is denoted as an “optical interconnection”. In the neural probe 100, 100′, lines for an optical interconnection and an electrical connection may be designed without mutual physical, optical, and electrical interference. For example, a line (the optical waveguide 133) for an optical interconnection and a line (the electric wire 143) for an electrical connection may be disposed at different heights on the basis of the neural probe substrate. That is, the line (the optical waveguide 133) for an optical interconnection and the line (the electric wire 143) for an electrical connection may be intersected without overlapping each other because the lines are not present on the same plane.

The neural probe substrate 110 may be made of a silicon (Si) or glass-based material having high physical strength. For example, a silicon (Si) or glass-based neural probe substrate has higher mechanical strength than a polyimide (PI) neural probe substrate. However, a material for the neural probe substrate according to an embodiment of the present disclosure is not limited to the example.

The neural probe substrate 110 functions to support the optical waveguide 133 so that the optical waveguide 133 disposed on the neural probe substrate 110 may transmit light emitted from an optical element to a target nerve cell through the optical output port 132. Specifically, the optical waveguide 133 is present on the neural probe substrate 110. An optical signal that is emitted from the optical element enters the optical waveguide 133 through the optical input port 131, passes through the inside of the optical waveguide 133, and reaches a target nerve cell through the optical output port 132.

A material for the optical waveguide 133 is preferably a material that has a high refractive index and that transmits visible light. For example, SiN, SiO₂, SiON, polymer, SU-8, or parylene may be used as a material for the optical waveguide 133. The top of the optical waveguide 133 may be exposed to the air, but may have a clad layer 160 for a light propagation loss reduction effect. SiN, SiO₂, SiON, polymer, PDMA, SU-8, or parylene may also be used as a material for the clad layer 160, but a material that is different from the material of the optical waveguide 133 and that has a lower refractive index needs to be used. The optical input port 131 or the optical output port 132 may be formed of diffracting grating, and may be a mirror surface. For example, the optical input port 131 or the optical output port 132 may be a 45-degree mirror surface. In this case, the 45-degree mirror surface changes the direction of an optical signal into a direction perpendicular to the direction of progress of the optical signal.

In the neural probe 100 of FIGS. 1A and 1B, a combination body of the protrusion part 112, the optical output port 132, and the first electrode 141 may be denoted as a probe part 120. That is, the probe part 120 includes the protrusion part 112, the optical output port 132, and the first electrode 141.

As described above, the first electrode 141 capable of receiving an electrophysiological signal generated from a nerve may be integrated in the probe part 120. The number of first electrodes 141 or second electrodes 142 may be one or plural like the optical signal port 130. The first electrode 141 transmits a neural signal collected by the second electrode 142. Each of the second electrodes 142 transmits a collected neural signal to a circuit, a terminal, or a system capable of finally analyzing a signal through an electrical method (e.g., wired communication) or wireless communication.

Platinum (Pt), gold (Au), titanium (Ti), titanium nitride (TiN), tungsten (W), molybdenum (Mo), iridium (Ir), iridium oxide (IrOx), platinum-iridium (Pt—Ir), silicon (Si), a carbon-based material (e.g., carbon nanotube or graphene), a polymer electrode (e.g., PEDOT), ruthenium oxide (RuOx), or stainless steel may be used as a material for the electrodes 141 and 142. In the neural probe substrate 110, the thickness of the probe part 120 or the protrusion part 112 may be different from the thickness of another part of the neural probe substrate 110. For example, an area (e.g., the base section 111) of the neural probe substrate 110, which is receive in the neural probe interface device 200, 300, 470, may be formed to be relatively thick in order to assign a physically robust characteristic. The probe part 120 or the protrusion part 112 that is inserted (e.g., pierced) into a biological tissue, specifically, a nerve may be formed to be relatively thin in order to minimize damage to the nerve.

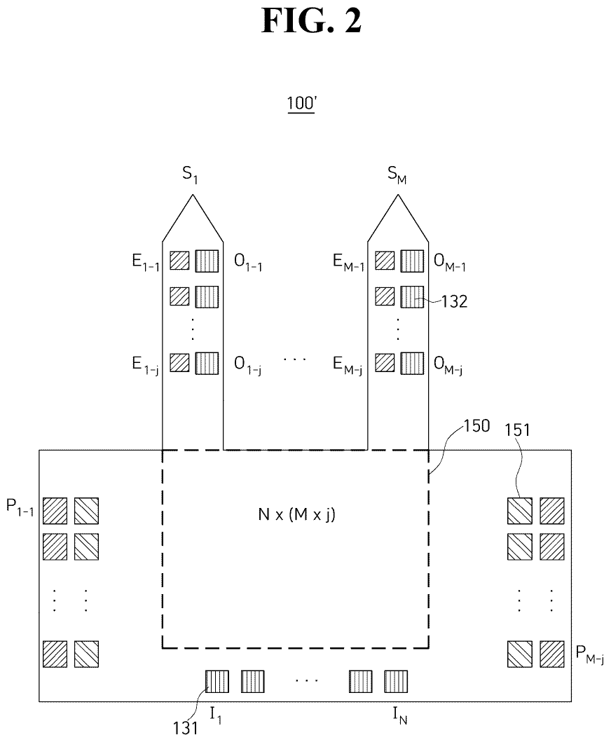

FIG. 2 is a diagram illustrating an embodiment of the neural probe in which an optical switch circuit has been integrated. An optical switch circuit 150 (may be denoted as an “optical switch”) that is integrated in a neural probe 100′ of FIG. 2 is an N*(M*j) switch (“*” refers to a multiplication symbol). In this case, N indicates the number of optical input ports, M indicates the number of probe parts, and j indicates the number of optical output ports per probe part.

As illustrated in FIG. 2, an optical interconnection between the optical input port 131 (I_{N}) and the optical output port 132 (O_{M-j}) may be dynamically changed by the N×(M×j) optical switch circuit. That is, a connection structure and a connection state between the optical input port 131 (I_{N}) and the optical output port 132 (O_{M-j}) may be changed in real time by the optical switch circuit 150.

A third electrode 151, that is, an electrode that controls the optical switch circuit 150, may be disposed on the upper part of the neural probe 100′ along the second electrode 142 (P_{M-j}). In contrast, the optical switch circuit 150 may be manually operated by the selection of the optical input port 131 (I_{N}) or depending on an input wavelength without control of the third electrode 151.

The number of optical input port 131 (I_{N}) can be reduced because the optical switch circuit 150 is integrated at the center of the neural probe 100′ as described above. In this case, a degree of difficulty of an optical interconnection can be reduced and a total volume of the neural probe 100′ can be reduced because the number of connection media required between the neural probe 100′ and an external light source is reduced. The optical switch circuit 150 may be implemented according to various methods, such as a phased-array method, an arrayed waveguide grating (AWG) method, a Mach-Zehnder interferometer (MZI) method, a ring resonator method, and a directional coupler method. Various function elements may be used in such an implementation.

FIG. 3 is a schematic diagram of a cross section of the neural probe.

FIG. 3 is a schematic diagram of a cross section of the neural probe 100 or the neural probe 100′. As illustrated in FIG. 3, on the basis of a contact surface of the neural probe substrate 110 and the clad layer 160, the heights of an optical waveguide core 133a and an electrode (e.g., the third electrode 151 or the first electrode 141) may be different. Furthermore, the heights of the first electrode 141 and the third electrode 151 may also be different. The optical waveguide core 133a and the electrode 141, 151 can be intersected without overlapping each other when a connection line (e.g., optical interconnection or electrical connection) is designed because the heights of the optical waveguide core and the electrode are different as described above. Accordingly, the area of the neural probe 100, 100′ can be efficiently used. An electrical connection may be made possible because the top of the clad layer 160 of the first electrode 141 or the second electrode 142, which requires an electrical connection with the outside or is necessary to measure a neural signal, is exposed to the air in the clad layer 160 (i.e., an insulator layer) for insulating the top of the electrode. However, other wire parts may be used without any change because the other wire parts are cover with the clad layer 160 without any change.

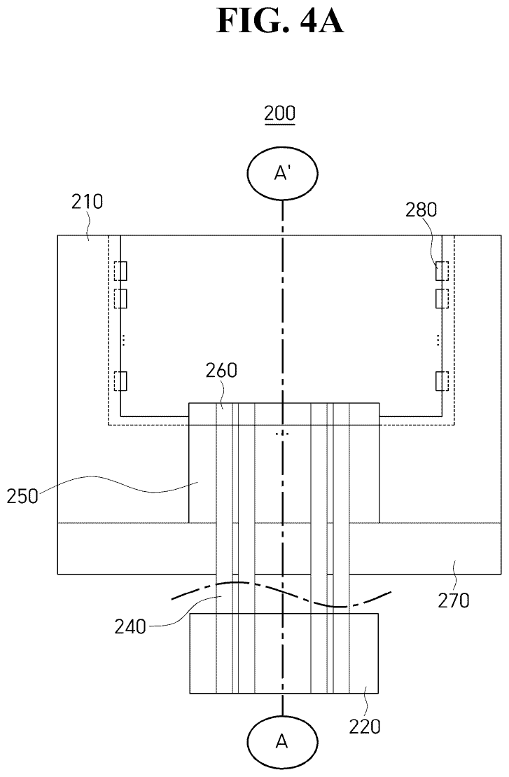

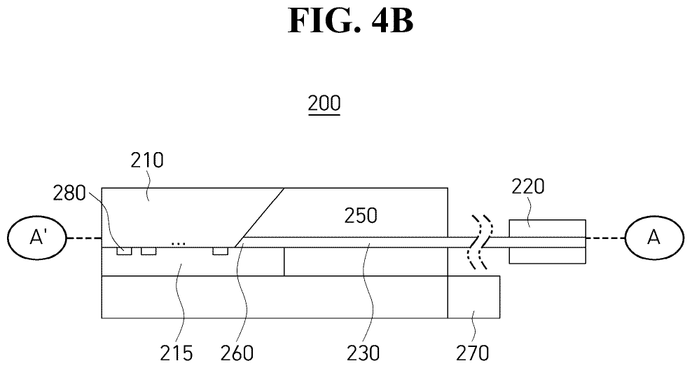

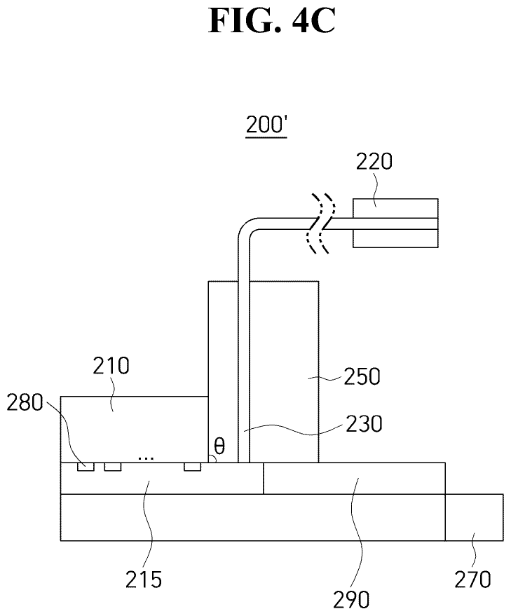

FIGS. 4A to 4C are diagrams illustrating a construction of an optical fiber-based neural probe interface device. FIG. 4A is a plan view of the optical fiber-based neural probe interface device. FIG. 4B is a cross-sectional view of a section taken along line A-A′ in FIG. 4A.

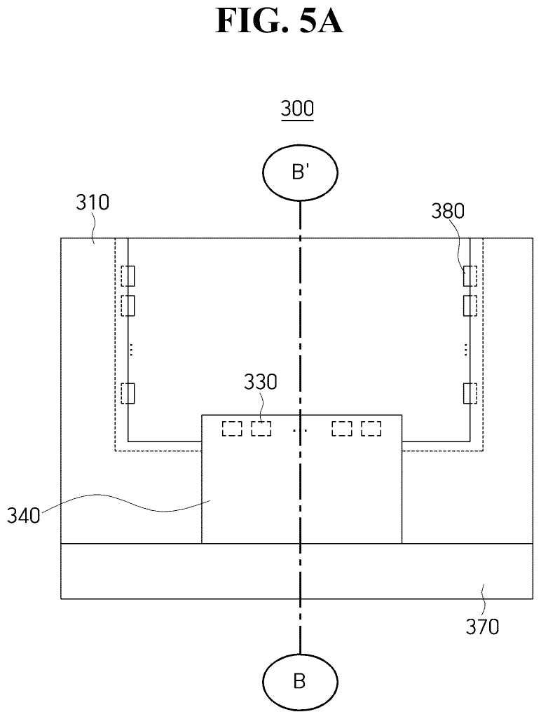

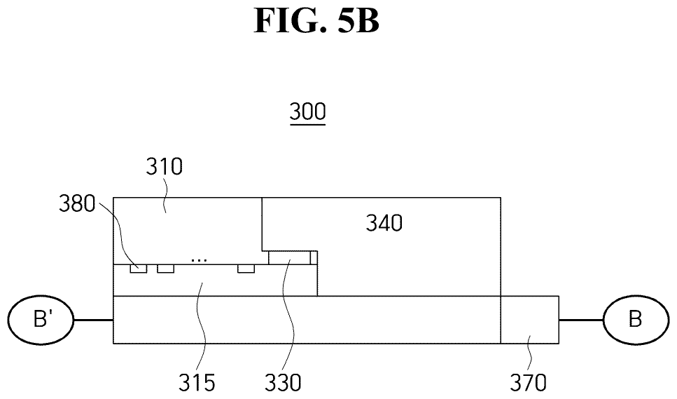

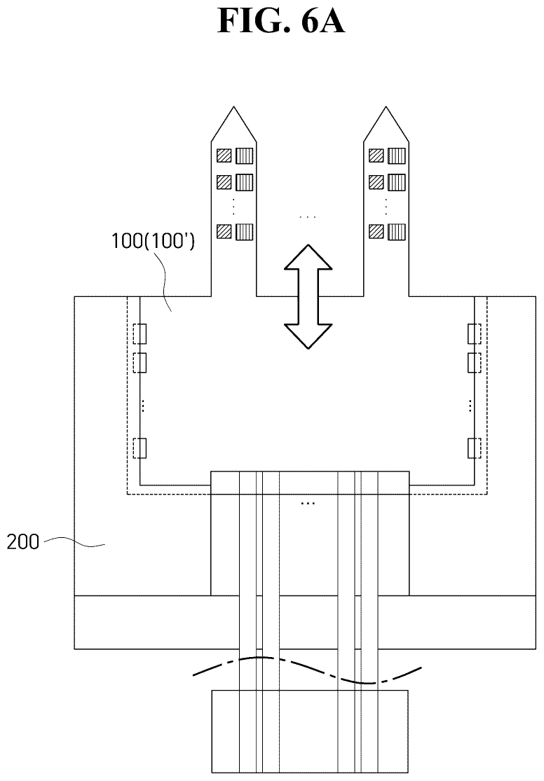

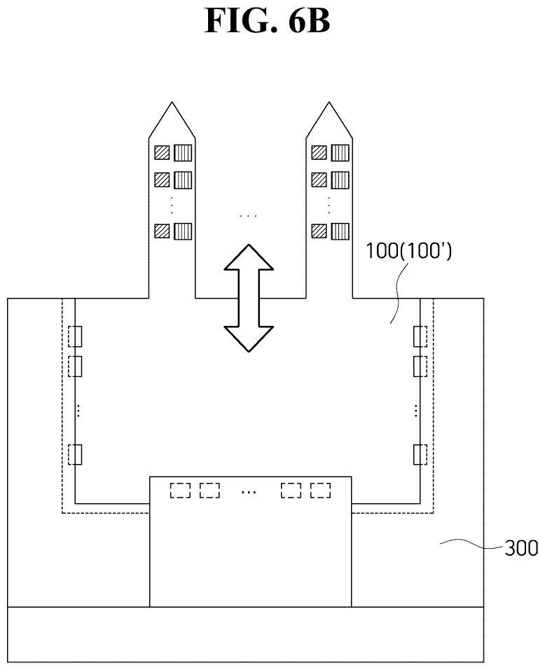

FIGS. 4A and 4B are construction diagrams of an optical fiber-based neural probe interface device 200 (hereinafter referred to as “neural probe interface device”) that implements the reception and optical/electrical connection of the neural probe 100, 100′ in order to use the neural probe 100, 100′ in experiments. The neural probe interface device 200 includes a neural probe holder 210 capable of receiving and fixing a neural probe. The neural probe holder 210 is constructed to be fixed by physical pressure of a neural probe reception part 215 that surrounds the edge of the neural probe. In FIG. 4A, a dotted line indicates the boundary line of the neural probe reception part 215 and a space (FIGS. 5A, 6A, and 6B are also the same). The neural probe holder 210 includes an electrode connection part 280 that enables an electrical connection of the second electrode 142 disposed on the upper part of the neural probe. An electrical signal from a nerve, which is received by the first electrode 141 of the neural probe, is transmitted to the second electrode 142. The electrical signal is transmitted to the electrode connection part 280 of the neural probe interface device 200, which comes into contact with the second electrode 142. Finally, the electrical signal is transmitted to a signal analyzer and a PC through an external electrode interface 270 (may be denoted as an “electrode connector”). If the optical switch circuit 150 is integrated in the neural probe, power and a control signal for control of the optical switch circuit 150 may be input to the electrode connection part 280 and the third electrode 151 that comes into contact with the electrode connection part 280 through the external electrode interface 270. The electrode connection part 280 of the neural probe interface device 200 is electrically connected to the external electrode interface 270. For example, an FFC cable connector may be used as the external electrode interface 270. An optical signal for neural stimulation/suppression from an external light source is transmitted through an optical fiber 230. The optical signal may be made to be incident on a specific optical input port 131 on the upper part of the neural probe by connecting an optical path from the external light source to the specific optical fiber 230 of an external optical fiber interface 220 (may be denoted as an “optical connector”). In this case, an optical interconnection between the optical fiber 230 and the optical input port 131 or optical output port 132 of the neural probe may be implemented by using a phenomenon in which the direction of progress of the optical signal is changed by total reflection of light by forming a mirror surface having a specific angle at the end of the optical fiber 230 within an optical fiber block 250. For example, the mirror surface may be formed to have an angle of 35 degrees to 50 degrees. Furthermore, special coating for increasing reflection efficiency may be added to the mirror surface. A 90-degree optical path transformer, diffracting grating, or a micro lens may be used as a method of changing the direction of progress of the optical signal instead of forming the mirror surface.

The optical fiber array block 250 (may be denoted as an “optical fiber block”) may have a recess area so that a lower area of an optical interconnection part 260 is exposed to the air. As the neural probe is connected to the recess area, an optical interconnection between the optical fiber 230 and the optical input port 131 of the neural probe can be implemented. The optical fiber array 240 is extended by a required length. The ends of the optical fibers 230 on a side opposite to the optical fiber array block 250 may have a form in which the ends are combined by the external optical fiber interface 220, such as an MT ferrule, or may be constructed in a form in which each optical fiber has each optical fiber connector. In such a form of the optical interconnection, all of the optical fiber block 250, the optical fiber array 240, and the external optical fiber interface 220 may be constructed as one element or module in addition to a form in which the optical fiber block 250, the optical fiber array 240, and the external optical fiber interface 220 are connected.

FIG. 4C is a diagram for describing an embodiment different from the embodiment of FIG. 4B in a method of making an optical signal incident on the optical input port 131. In a neural probe interface device 200′ according to the embodiment of FIG. 4C, a method of transmitting an optical signal to the optical input port 131 by making the optical fiber 230 included in the optical fiber array block 250 have a specific angle (e.g., an angle of 90 degrees or less, θ≤90°) with respect to the top of the neural probe 100, 100′ inserted into the neural probe reception part 215 is adopted instead of forming the mirror surface having a specific angle at the end of the optical fiber 230. In other words, in FIG. 4C, the optical fiber 230 close to the optical input port 131 has a difference of a specific angle compared to the optical fiber 230 of FIG. 4B.

The neural probe interface device 200′ according to such a method has an advantage in that the location of the external optical fiber interface (or the optical connector) 220 can be changed by using the flexibility of the optical fiber 230. In the embodiment of FIG. 4B, the space of the neural probe reception part 215 is secured through the recess structure of the optical fiber block 250. In contrast, in the embodiment of FIG. 4C, a separate spacer 290 is disposed under the optical fiber block 250 in order to secure the space of the neural probe reception part 215.

FIGS. 5A and 5B are diagrams illustrating a construction of an optical element-based neural probe interface device. FIG. 5A is a plan view of the optical element-based neural probe interface device. FIG. 5B is a cross-sectional diagram of a section taken along line B-B′ in FIG. 5A.

FIGS. 5A and 5B illustrate an optical element-based interface device 300 (hereinafter denoted as a “neural probe interface device”) that implements the reception, optical connection, and electrical connection of the neural probe 100, 100′ in order to use the neural probe in experiments. In the embodiments of FIGS. 4A and 4B, the optical fiber is used. In contrast, in the neural probe interface device according to the embodiment of FIGS. 5A and 5B, an optical element array 340 in which optical elements 330 that directly generate an optical signal are aligned at predetermined intervals is used. In this case, the optical element 330 may be a light-emitting element, such as a vertical cavity surface emitting laser (VCSEL), a distributed feedback laser (DFB laser), a Fabry-perot laser, a quantum cascade laser, a quantum dot laser, an LED, a micro-LED, a mini-LED, or an OLED. Some of the optical elements may be formed of light-receiving elements, such as an Si PD, a Ge PD, and an APD. In the optical element array 340 of FIGS. 5A and 5B, one type of optical elements 330 may be aligned at predetermined intervals, and two types or more of optical elements 330 may be aligned in a form in which the optical elements 330 are disposed at required locations. The neural probe interface device 300 may transmit an electrical neural signal received by the neural probe 100, 100′ to the outside through an external electrode interface (or connector) 370, and may also receive power and a control signal for driving the optical elements 330 from the outside. Functions of a neural probe holder 310, a neural probe reception part 315, and an electrode connection part 380 are the same as those of the neural probe holder 210, the neural probe reception part 215, and the electrode connection part 280 in FIGS. 4A and 4B, respectively.

FIGS. 6A and 6B are exemplary diagrams of a combination of the neural probe and the neural probe interface device.

As illustrated in FIGS. 6A and 6B, the neural probe 100, 100′ may be combined with the neural probe interface device. In this case, the neural probe interface device may be the optical fiber-based neural probe interface device 200 illustrated in FIGS. 4A and 4B, and may be the optical element-based neural probe interface device 300 illustrated in FIGS. 5A and 5B. In this specification, a device having a form in which the neural probe 100 or 100′ and the neural probe interface device 200 or 300 are combined is denoted as a “neural probe device”.

The neural probe 100, 100′ may be combined with the reception part 215, 315 of the neural probe interface device 200, 300 by sliding the neural probe 100, 100′ in a direction indicated by an arrow in FIGS. 6A and 6B. The neural probe 100, 100′ that has been used may be separated from the neural probe interface device. The reception part 215, 315 of the neural probe interface device is designed to have a size according to the size of the base section 111 of the neural probe 100, 100′ so that an optical interconnection and an electrical connection are accurately performed upon reception.

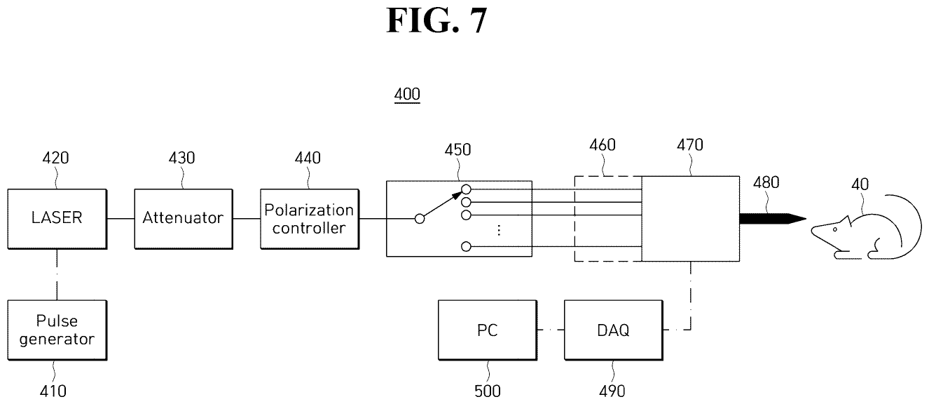

FIG. 7 is a diagram of a biological experiment system using the neural probe. In FIG. 7, a slid line refers to an optical interconnection section, and a dash-dotted line refers to an electrical connection section.

In FIG. 7, a biological experiment system 400 includes a signal generator 410, a light source (LASER) 420, an attenuator 430, a polarization controller 440, an optical switch 450, an optical fiber array connector 460, a neural probe interface device 470, a neural probe 480, a data acquisition system (DAQ) 490, and a computer (PC) 500. The neural probe interface device 470 may be the neural probe interface device 200. The neural probe 480 may be the neural probe 100 of FIGS. 1A and 1B or the neural probe 100′ of FIG. 2. The biological experiment system 400 illustrated in FIG. 7 is an embodiment. The components of the biological experiment system 400 according to an embodiment of the present disclosure are not limited to the embodiment of FIG. 7, and a component may be added to the biological experiment system 400 or some of the components of the biological experiment system 400 may be changed or deleted.

An optical signal that is output from the light source 420 is output in the form of a pulse at predetermined interval by the signal generator 410. The optical signal may be input to the optical fiber 230 of the neural probe interface device 200. A required intensity of the optical signal is adjusted by the attenuator 430. The optical signal is processed into polarization suitable for a targeted polarization state by the polarization controller 440. The biological experiment system 400 of FIG. 7 may transmit an optical signal by selecting a port, that is, a connection target, among the optical input ports 131, for example, N optical input ports that are present in the nerve probe 480 by using the optical switch 450, for example, a (1*N) optical switch. The required number of optical fibers is connected to the output stage of the optical switch 450. An optical signal that is input to the optical switch 450 is transmitted through a selected specific output optical fiber. The optical fibers are subjected to an optical interconnection with the neural probe interface device 470 through the optical fiber array connector 460. An optical signal that is input to a specific optical fiber 230 is incident on the optical input port 131 of the neural probe 100, 100′ that is optically connected to the neural probe interface device 470, and is then transmitted to a nerve cell of a biological experiment target 40 (e.g., a rat) through a target optical output port 132. At this time, an electrical neural signal that is generated from a plurality of nerve cells of the biological experiment target 40 is obtained through the first electrode 141, that is, an electrode for extracting a neural signal, of the neural probe 100, 100′. The electrical neural signal is transmitted to the second electrode (or the electrode pad) 142 that is electrically connected to the first electrode 141. Thereafter, the electrical neural signal is transmitted to the data acquisition system 490 through the electrode connection part 280 and external electrode interface (or connector) 270 of the neural probe interface device 470. Finally, the electrical neural signal is transmitted to the computer 500. The computer 500 analyzes an active pattern of a plurality of neural signals.

If the optical element-based neural probe interface device 300 is used, an experiment device in an optical interconnection section from the light source 420 to the optical fiber array connector 460 in the biological experiment system 400 of FIG. 7 may be omitted. A biological experiment system 400′ (not illustrated) may be implemented in a way to directly electrically connect a signal generation/input device for controlling an optical element to the neural probe interface device 300.

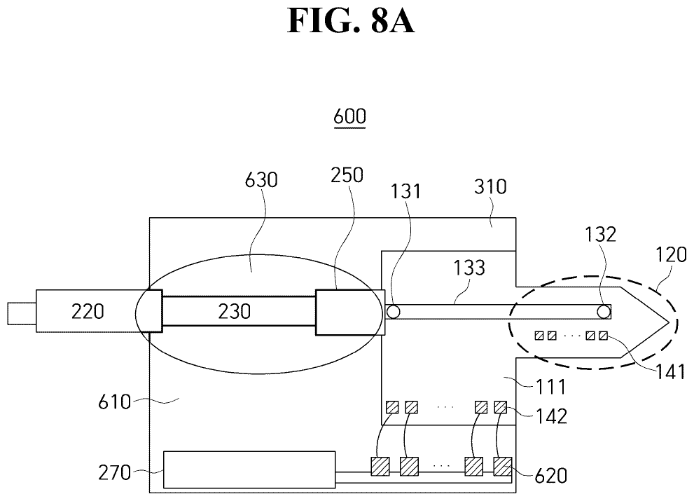

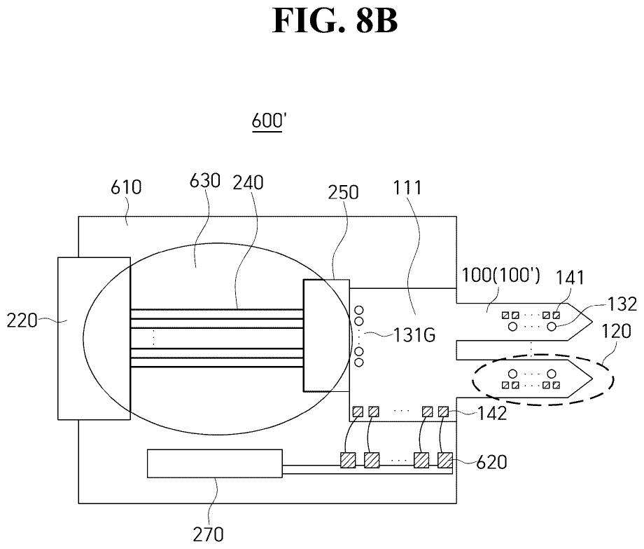

FIGS. 8A and 8B are exemplary diagrams of a neural probe device 600, 600′ in which the neural probe and an interposer are combined.

FIGS. 8A and 8B illustrate examples in which the neural probe devices 600 and 600′ are constructed by combining an interposer 610 with the neural probes 100 and 100′ instead of the detachable neural probe interface devices 200 and 300. The neural probe 100, 100′, the optical fiber 230, the external optical fiber interface (or optical connector) 220, the optical fiber block 250, and the external electrode interface (or electrode connector) 270 are mounted on a PCB or a glass-based neural probe substrate that plays a role as the interposer 610 in a form in which all of the neural probe 100, 100′, the optical fiber 230, the external optical fiber interface (or optical connector) 220, the optical fiber block 250, and the external electrode interface (or electrode connector) 270 have been physically fixed. For the physical fixing of each element, resin 630 may be applied on the interposer 610.

As illustrated in FIG. 8A, when each of the number of probe parts 120, the number of optical input ports 131, and the number of optical output ports 132 is one, the optical connector 220 may be connected to only one optical fiber (or waveguide) 230. Accordingly, a ferrule connector may also be applied as the optical connector 220 in addition to various optical connectors, such as FC/LC/ST/SC/MTRJ to which one optical connector may be connected.

The connection of the nerve probe 100 and the optical fiber 230 (or the waveguide) may be implemented by using the optical fiber block 250 having the mirror surface as illustrated in FIG. 4. Various optical path transformers may also be used instead of the optical fiber block 250. For example, a direct optical wiring (DOW) technique, a polymer optical path transformer using a mask transfer method, or an optical path transformer using a mirror surface or a lens may be used as the optical path transformer.

The electrodes 141 and 142 of the probe part 120 and base section 111 of the neural probe 100 are electrically connected. An array of the second electrodes 142 (a second electrode array) of the base section 111 is electrically connected to a fourth electrode 620, that is, an electrode on the interposer 610. In this case, an electrical connection between the neural probe 100 and the interposer 610 may be implemented by a connection method using wire bonding in addition to a contact method in which electrodes physically come into contact with each other. The fourth electrode 620, that is, an electrode on the interposer 610, is connected to the electrode connector 270 through an electrode line on the interposer 610. An external signal may be obtained or communication with an external analysis system may be performed through the electrode connector 270.

As illustrated in FIG. 8B, when each of the number of probe parts 120, the number of optical input ports 131, and the number of optical output ports 132 of the nerve probe 100, 100′ is plural, the number of channels capable of an optical interconnection by the optical connector 220, the optical fiber array 240, and the optical fiber block 250 (or the optical path transformer) is also increased. In this case, various types of optical connectors, such as a ferrule connector (FC), a lucent connector (LC), a square connector (SC), a straight tip (ST), a mechanical transfer registered jack (MT-RJ), and a ferrule, may be used at the end of each optical fiber 230 (or waveguide) as the optical connector 220. An external optical fiber interface (or optical connector) having a form in which several elements are combined, like a mechanical transfer (MT) ferrule, may also be used. A connection between the nerve probe 100, 100′ and the optical fiber 230 (or the waveguide) and a connection between the neural probe 100, 100′ and the electrode of the interposer 610 is the same as the case in which each of the number of probe parts 120, the number of optical input ports 131, and the number of optical output ports 132 is one (FIG. 8A).

Although the present disclosure has been described with reference to the preferred embodiments, those skilled in the art may understand that the present disclosure may be modified and changed in various ways without departing from the spirit and scope of the present disclosure written in the claims.

### DESCRIPTION OF REFERENCE NUMERALS

40: subject of biological experiments
100: neural probe
110: neural probe substrate
111: base section
112: protrusion part
120: probe part
130: optical signal port
131: optical input port
132: optical output port
133: optical waveguide
133a: optical waveguide core
141: first electrode (electrode for extracting neural signal)
142: second electrode (electrode pad)
143: electric wire
150: optical switch (optical switch circuit)
151: third electrode (electrode for controlling optical switch)
160: clad layer
200: neural probe interface device
210: neural probe holder
215: neural probe-receiving part
220: external optical fiber interface (optical connector)
230: optical fiber 240: optical fiber array
250: optical fiber block
260: optical connection part
270: external electrode interface (electrode connector)
280: electrode connection part
300: neural probe interface device
310: neural probe holder
315: neural probe-receiving part
330: optical element (optical connection part)
340: optical element array
370: external electrode interface (electrode connector)
380: electrode connection part
400: biological experiment system
410: signal generator 420: light source
430: attenuator
440: polarization controller 450: optical switch
460: optical fiber array connector
470: neural probe interface device
480: neural probe
490: data acquisition system 500: computer
600: neural probe device 610: interposer
620: fourth electrode (interposer electrode)
630: resin

What is claimed is:

1. A neural probe that collects a neural signal generated from neurons based on an optogenetics technique, the neural probe comprising:
a neural probe substrate;
one or more optical input ports disposed in the neural probe substrate and configured to receive an optical signal from an outside;
one or more optical output ports disposed in the neural probe substrate and configured to transmit the optical signal to neurons;
one or more first electrodes configured to receive the neural signal; and
one or more second electrodes configured to receive the neural signal from the first electrode through an electric wire and to transmit the neural signal to the outside by using any one of a wired communication method and a wireless communication method.

2. The neural probe of claim 1, wherein:
the neural probe substrate comprises a base section and a protrusion part, and
a thickness of the protrusion part is a thickness or less of the base section.

3. The neural probe of claim 2, wherein the optical input port is mounted on the base section.

4. The neural probe of claim 2, wherein the optical output port is mounted on the protrusion part.

5. The neural probe of claim 2, wherein the first electrode is mounted on the protrusion part.

6. The neural probe of claim 2, wherein the second electrode is mounted on the base section.

7. The neural probe of claim 1, further comprising an optical waveguide disposed in the neural probe substrate and configured to transmit the optical signal to the optical output port.

8. The neural probe of claim 7, wherein the optical waveguide comprises any one material, among SiN, SiO₂, SiON, polymer, SU-8, and parylene.

9. The neural probe of claim 7, wherein the optical waveguide and the electric wire are disposed at different heights in relation to the neural probe substrate.

10. The neural probe of claim 1, further comprising an optical switch circuit disposed in the neural probe substrate and configured to transmit the optical signal to the optical output port,
wherein the optical switch circuit dynamically changes an optical interconnection between the optical input port and the optical output port in response to a control signal.

11. A neural probe interface device that receives a neural probe for collecting a neural signal generated from neurons based on an optogenetics technique and that is connected to the neural probe,
wherein the neural probe comprises:
a neural probe substrate;
one or more optical input ports disposed in the neural probe substrate and configured to receive an optical signal from an outside;
one or more optical output ports disposed in the neural probe substrate and configured to transmit the optical signal to neurons;
one or more first electrodes configured to receive the neural signal; and
one or more second electrodes configured to receive the neural signal from the first electrode through an electric wire and to transmit the neural signal to the outside by using any one of a wired communication method and a wireless communication method, and
the neural probe interface device comprises:
a neural probe holder configured to receive and fix the neural probe;
an electrode connection part connected to the second electrode and configured to receive the neural signal from the second electrode; and
an external electrode interface configured to transmit the neural signal to an external signal analyzer or a computer.

12. The neural probe interface device of claim 11, further comprising an optical connector configured to receive an optical signal for neural stimulation or suppression from an external light source and to make the optical signal incident on the optical input port of the neural probe through an optical fiber.

13. The neural probe interface device of claim 11, further comprising an optical element array comprising optical elements aligned at predetermined intervals,
wherein an optical signal emitted from the optical element is incident on the optical input port of the neural probe.

14. A biological experiment system that performs biological experiments by using a neural probe for collecting a neural signal generated from neurons based on an optogenetics technique, the system comprising:
a light source configured to output an optical signal;
a signal generator configured to output the optical signal in a form of a pulse at predetermined interval;
an optical switch configured to transmit the optical signal by selecting one or more of optical input ports of the neural probe by setting;
a neural probe configured to receive the optical signal, transmit the optical signal to neurons of a biological experiment target, and receive the neural signal from the neurons;
a data acquisition system configured to collect the neural signal; and
a computer configured to generate results of analysis of an active pattern by analyzing the neural signal collected by the data acquisition system.

15. The biological experiment system of claim 14, further comprising an attenuator configured to adjust an intensity of the optical signal output from the light source.

16. The biological experiment system of claim 14, further comprising a polarization controller configured to control polarization of the optical signal output from the light source.

17. The biological experiment system of claim 14, further comprising:
an optical fiber array connector configured to receive the optical signal from the optical switch through a selected optical fiber; and
a neural probe interface device connected to the optical fiber array connector and configured to transmit the optical signal received from the optical fiber array connector from the optical switch to an optical input port of the selected neural probe.

18. The biological experiment system of claim 14, wherein the neural probe comprises:
a neural probe substrate;
one or more optical input ports disposed in the neural probe substrate and configured to receive the optical signal;
one or more optical output ports disposed in the neural probe substrate and configured to transmit the optical signal to neurons;
one or more first electrodes configured to receive the neural signal; and
one or more second electrodes configured to receive the neural signal from the first electrode through an electric wire and to transmit the neural signal to the outside by using any one of a wired communication method and a wireless communication method.

19. The biological experiment system of claim 17, wherein the neural probe interface device comprises:
a neural probe holder configured to receive and fix the neural probe;
an electrode connection part configured to receive a neural signal collected from the biological experiment target by the neural probe from a second electrode of the neural probe; and
an external electrode interface configured to transmit the neural signal to an external signal analyzer or a computer.
